(19) United States
(12) Patent Application Publication
(10) Pub. No.: US 2010/0305576 A1
(43) Pub. Date: Dec. 2, 2010

(54) SUTURE ANCHORING INSTRUMENT

(75) Inventors: Joe W. FERGUSON, Memphis, TN (US); Joseph R. WOODARD, Memphis, TN (US)

Correspondence Address: CHESTER G. MOORE;Baker, Donelson, Bearman, Caldwell & Berkowitz, PC, 3 Sanctuary Boulevard, SUITE 201, Mandeville, LA, 70471, US

(73) Assignee: WRIGHT MEDICAL TECHNOLOGY, INC., Arlington, TN (US)

(21) Appl. No.: 12/474,515

(22) Filed: May 29, 2009

(51) Int. Cl. A61B 17/58; A61B 17/04

(52) U.S. Cl. 606/104; 606/232

(57) ABSTRACT

An installation tool is provided for producing a threaded hole and deploying an anchoring device into the threaded hole, while retaining control over associated sutures. The tool comprises a handle, a longitudinally-extending shaft removably engaged with the handle, an anchor removably engaged with the shaft, at least one suture threaded through the anchor and engaged with the handle, and a tap sleeve removably engaged with the shaft and which accommodates the anchor, at least a portion of the shaft, and at least a portion of the at least one suture.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON COMPACT DISC

Not applicable.

### BACKGROUND OF THE INVENTION

1. Field of the Invention

The present invention relates generally to surgical devices for forming holes in bone, and for deploying and installing anchoring devices into said holes. More particularly, the invention relates to suture anchor installation tools. Still more particularly, the invention relates to devices including retention means for organizing and holding the free ends of at least one suture or the like extending from an anchoring device, and such needle-like elements as may be attached thereto.

2. Description of Related Art

Suture anchors for positioning an end or intermediate portion of a length of suture within a preformed hole in bone, as well as suture anchor installation tools for deploying such suture anchors, are known in the art. Some of these tools include a handle adapted to hold portions of free suture ends extending from the suture anchor, and any needles that may be attached to such free ends.

The principal purpose of each of the foregoing devices is to provide attachment means for positioning—and for substantially fixedly attaching—a portion of a length of suture to a piece of bone. For a variety of reasons, other attachment means—including nails, screws, and staples—are not satisfactory because: 1) they may cause undesirable damage during installation; 2) they may loosen or fail when subjected to normal post-operative stresses; and 3) they are not adapted to reliably and fixedly hold a length of suture. Thus, suture anchors designed to permanently and fixedly locate a portion of at least one length of suture within a preformed hole in bone have been developed.

Suture anchors generally possess receiving means (including, but not limited to an eyelet, slots, or recesses) for at least one length of suture, whereby the suture may be held by the suture anchor. The relatively confined space in which suture anchors are deployed, and the difficulties associated with threading a length of suture through a suture anchor after it has been inserted into a bone, make post-insertion threading of suture undesirable and impractical. Thus, suture anchors are generally threaded with at least one length of suture prior to their being inserted into a bone. Consequently, the free suture ends extending from the suture anchor must be managed so that they do not become tangled during insertion of the suture anchor.

Along with the development of suture anchors came the development of drivers for installing the suture anchors. Generally, suture anchor drivers provide means for attaching to and engaging with the anchor (e.g., a socket, pins, or other such features on the distal end of the driver, mated to corresponding features on the proximal end of the anchor), conveying the anchor to a desired location, and driving the anchor into the bone. To control or manage the free ends of suture threaded through and extending from the anchor, which otherwise would become tangled and/or obscure the operative field, various methods have been devised to releasably hold the free ends.

While the prior art discloses, generally, pre-threaded suture anchors and suture anchor drivers, those devices necessitate either: 1) a self-tapping suture anchor, wherein the suture anchor itself is designed to cut the insertion hole into the bone as the anchor is inserted; or 2) a separate, dedicated device for producing a hole in a bone, suitable for receiving the suture anchor (e.g., a threaded bone tap—a tool for forming an internal screw thread in bone).

Self-tapping suture anchors may be undesirable because they are resisted by the bone chips formed as they are driven into the bone, and because they cannot be made with polymeric materials. As a self-tapping suture anchor is driven into a bone, the driving force required to advance the anchor increases as bone chips accumulate and resist advancement of the anchor. The user must either back the self-tapping suture anchor out from the hole and clean out the chips formed, or risk: 1) jamming the anchor in an undesirable location (e.g., at an inappropriate depth); 2) fracturing the anchor from applying excessive force; or 3) stripping the newly-cut threads on the inner surface of the hole. A self-threading anchor must also be sufficiently sturdy to withstand the forces required to produce a threaded hole, thus foreclosing the possibility of using certain materials (e.g., biodegradable materials, certain plastics, etc.) to make self-tapping anchors. Finally, the axial and torsional forces required to create a hole in a bone are substantial. Because the suture anchor and driver are removably engaged over a comparatively small surface area, force sufficient to create a hole in a bone using a self-tapping suture anchor may also cause inadvertent disengagement of the suture anchor from the driver. This creates a hazard to medical personnel and patients alike.

A separate, dedicated bone tap is similarly undesirable because it must be separately sterilized, it is comparatively large (comprising at least a handle, shaft, and tap), it occupies valuable space amongst all the other surgical instruments needed during surgical procedures, and it involves unnecessary duplication of components (e.g., a separate handle for the tap). A separate, dedicated bone tap is also undesirable because the user must ensure that the hole diameter and thread profile produced by the chosen tap will be compatible with the suture anchor (e.g., thread diameter, pitch diameter). Otherwise, a substantial risk exists that the diameter and/or threads of the hole produced by the tap may not be suitable for use with the anchor.

### BRIEF SUMMARY OF THE INVENTION

In view of the foregoing, the primary object of the present invention is to provide a unitary suture anchor installation device, comprising suture and needle retention means releasably attachable to the device, along with tap means releasably attachable to the device and separate from the suture anchor itself.

Another object of the present invention is to provide a suture anchor installation device wherein: a) a tap for creating a threaded bone hole—an internal screw thread in bone—is releasably engaged; b) the tap is operated to create a threaded hole in a bone—a bone hole—then reversed; c) upon disengagement of the tap from the device, an anchor, at least one length of suture, and a shaft are exposed, the anchor being threaded by the at least one length of suture and also engaged by the shaft for transport to the bone hole; d) the anchor is inserted into the bone hole via a handle operating in association with the at least one length of suture and the shaft; and e) the handle comprises doors releasably engaged with the handle and which further comprise means for attaching free ends of the at least one length of suture and any objects attached thereto.

In one embodiment, an apparatus for inserting surgical implants is provided, comprising: a handle; a shaft extending along a longitudinal axis between first and second ends, wherein the shaft second end engages the handle; an anchor extending along a longitudinal axis between proximal and distal ends, wherein the anchor proximal end engages the shaft first end; and a tap sleeve extending along a longitudinal axis between proximal and distal ends, the tap sleeve having a blind bore with an opening at said tap sleeve proximal end and extending longitudinally toward said tap sleeve distal end, and the tap sleeve distal end comprising a tap; wherein the tap sleeve removably accommodates said anchor and at least a portion of the shaft first end via the tap sleeve blind bore.

In one aspect of this embodiment, the shaft further comprises at least one post. Preferably, the at least one post is cylindrical and projects perpendicular to the shaft longitudinal axis. More preferably, the shaft comprises first and second posts—which may or may not be cylindrical—projecting perpendicular to the shaft longitudinal axis and positioned about 180° from one another about the shaft longitudinal axis. In this aspect, the tap sleeve further comprises at least one notch, wherein the at least one notch is keyed to engage the at least one post. Preferably, the tap sleeve comprises first and second notches keyed to engage the first and second posts.

In another aspect of this embodiment, the apparatus further comprises at least one suture having first and second ends, the anchor further comprises threads, an eyelet, and at least one channel, and the shaft first end comprises at least one installation prong. Preferably, the at least one suture is threaded through the eyelet, and the at least one channel and the at least one installation prong are keyed to engage one another. More preferably, the anchor comprises first and second channels, and the shaft first end comprises first and second installation prongs, wherein the first and second channels and the first and second installation prongs are keyed to engage one another.

In another aspect of this embodiment, the handle comprises at least one cavity and at least one door, wherein the at least one door comprises attachment means for the at least one suture. In a preferred aspect, the attachment means for the at least one suture comprise a plurality of cleats arranged to engage the at least one suture. Preferably, the plurality of cleats are located on the at least one door inner surface. Also preferably, the at least one door is releasably engaged with the handle. More preferably, the handle comprises first and second cavities and first and second doors, wherein the first suture end is engaged with the plurality of cleats of the first door, and the second suture end is engaged with the plurality of cleats of the second door.

In yet another aspect of this embodiment, the first suture end further comprises a first needle attached thereto, and the second suture end further comprises a second needle attached thereto. and the first and second needles are located in the first and second cavities of the handle, respectively.

In another aspect of this embodiment, the shaft further comprises at least one furrow extending substantially from said shaft first end and toward shaft second end, whereby said at least one suture is directed toward the handle, and the handle further comprises at least one suture slot, whereby said at least one suture communicates with said at least one cavity. Preferably, the shaft comprises first and second furrows extending substantially from said shaft first end and toward shaft second end, whereby the first and second suture ends are directed along and within said first and second furrows, respectively, toward the handle, and the handle comprises first and second suture slots, whereby the first suture end communicates with the first cavity and the second suture end communicates with the second cavity.

In yet another aspect of this embodiment, the shaft second end is removably engaged with said handle, the said shaft first end may removably engage with said handle and said shaft second end further comprises at least one removal prong keyed to engage said at least one channel.

In a preferred aspect, an apparatus for inserting surgical implants is provided, comprising: a handle, comprising first and second cavities and first and second doors releasably engaged with the handle, each door comprising a plurality of cleats on the door inner surface, said handle further comprising first and second suture slots; a shaft extending along a longitudinal axis between first and second ends, wherein the shaft second end removably engages the handle and wherein the shaft first end may removably engage with the handle, wherein the shaft comprises first and second posts extending substantially perpendicular to the shaft longitudinal axis, wherein the shaft first end comprises first and second installation prongs, the shaft second end comprises first and second removal prongs, the shaft further comprises first and second furrows extending substantially from said shaft first end and toward shaft second end; an anchor extending along a longitudinal axis between proximal and distal ends, comprising threads, an eyelet, and first and second channels, wherein the anchor proximal end engages the shaft first end, and the first and second channels and first and second installation prong are keyed to engage one another; at least one suture having first and second ends, wherein the at least one suture passes through the eyelet, the first suture end further comprises a first needle and is engaged with the plurality of cleats of the first door, the second suture end further comprises a second needle and is engaged with the plurality of cleats of the second door, whereby the first and second needles are located in the first and second cavities, respectively; and a tap sleeve extending along a longitudinal axis between proximal and distal ends, the tap sleeve having a blind bore with an opening at said tap sleeve proximal end and extending longitudinally toward said tap sleeve distal end, the tap sleeve distal end comprising a tap, the tap sleeve proximal end comprising first and second notches keyed to engage first and second posts; whereby the first and second suture ends are directed along and within said first and second furrows, respectively, toward the handle, whereby the first suture end communicates with the first cavity and the second suture end communicates with the second cavity, wherein the tap sleeve removably accommodates said anchor, said at least one suture, and at least a portion of the shaft first end via the tap sleeve blind bore.

In a second embodiment, a method for attaching soft tissue to bone is provided, comprising the steps of providing an apparatus comprising: a handle, comprising first and second cavities and first and second doors releasably engaged with the handle, each door comprising a plurality of cleats on the door inner surface, said handle further comprising first and second suture slots; a shaft extending along a longitudinal axis between first and second ends, the shaft second end removably engaged with the handle; an anchor extending along a longitudinal axis between proximal and distal ends and comprising an eyelet, wherein the anchor proximal end engages the shaft first end; at least one suture having first and second ends, the suture being threaded through the eyelet, the first suture end comprising a first needle and the second suture end comprising a second needle, wherein the first suture end is engaged with the plurality of cleats of the first door and the second suture end is engaged with the plurality of cleats of the second door; and a tap sleeve extending along a longitudinal axis between proximal and distal ends, wherein the distal end comprises a tap, the tap sleeve further comprising a blind bore extending along the longitudinal axis, the tap sleeve removably attached to the shaft first end and accommodating the anchor, at least a portion of the at least one suture, and at least a portion of the shaft first end; forming a threaded hole in a bony structure with the tap of the tap sleeve removably attached to the shaft first end; removing the tap sleeve from the shaft first end; inserting the anchor into the threaded hole; and attaching soft tissue to said suture.

These and other objects of the invention are achieved by the provision and use of a novel suture anchoring device. In the preferred embodiment, the device is adapted to create a threaded hole in a bone and to deploy a suture anchor into the hole.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements.

FIG. 1 shows an embodiment of the present invention useful for producing a hole in a bone and for inserting suture anchors.

FIG. 2 is an enlarged fragmentary side view of the distal end of an embodiment of the present invention, with a portion of the tap sleeve cut away to show the shaft, suture anchor, and at least one suture.

FIG. 3 shows a perspective side view of an embodiment of the present invention after removal of the tap sleeve.

FIG. 4 shows the embodiment of FIG. 3, with both doors partially opened.

FIG. 5 is an enlarged fragmentary perspective view showing a bone hole being created with the an embodiment of the present invention.

FIG. 6 is an enlarged fragmentary perspective view showing a suture anchor being installed with an embodiment of the present invention.

FIG. 7 is a fragmentary perspective view showing a door opened to reveal at least one suture end connected thereto.

FIG. 8 is a side view of an embodiment of the present invention showing release of the door of FIG. 7 from the handle.

FIG. 9 shows an enlarged perspective side view of a door of an embodiment of the present invention.

FIG. 10 shows an enlarged, fragmentary, exploded view of the handle opening and anti-rotation sleeve element of an embodiment of the present invention.

FIG. 11A shows an enlarged side view of the shaft of an embodiment of the present invention, and FIG. 11B shows the shaft of FIG. 11A taken generally along line X in FIG. 11A.

FIG. 12 shows an enlarged perspective side view of the distal end of the embodiment of FIGS. 3 and 4.

FIG. 13 is an enlarged side view of an embodiment of the present invention during a stage of its use in a surgical procedure to insert a suture anchor.

FIG. 14 shows an enlarged side view of an embodiment of the present invention during a stage of its use in a surgical procedure to insert a suture anchor.

### DETAILED DESCRIPTION OF THE INVENTION

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

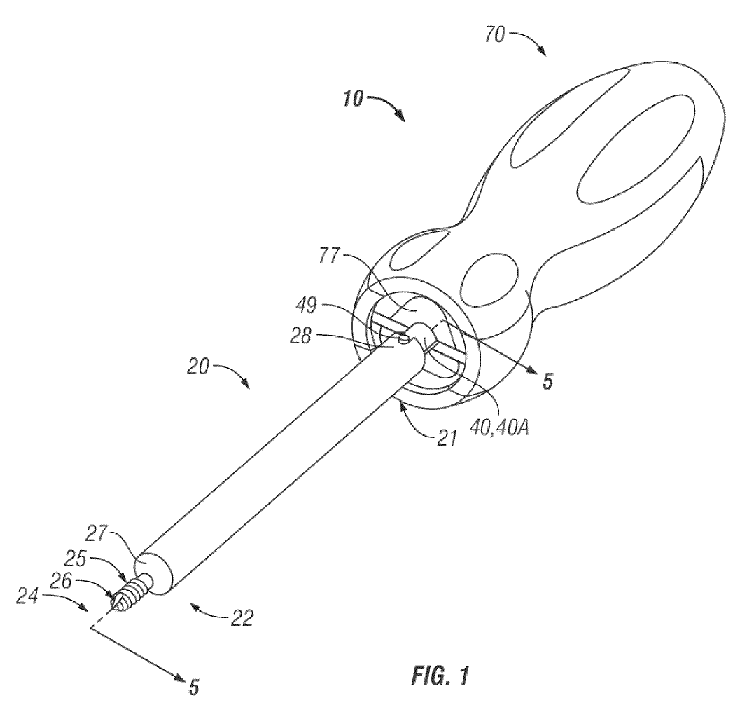

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs. Referring now to the drawings, where like elements are designated by like reference numerals, FIG. 1 illustrates an instrument (10) provided with handle (70) and removable tap sleeve (20) of the present invention. Instrument (10) comprises an elongated shaft (40) disposed within the tap sleeve (20) and removably attached to the distal end (72) of handle (70).

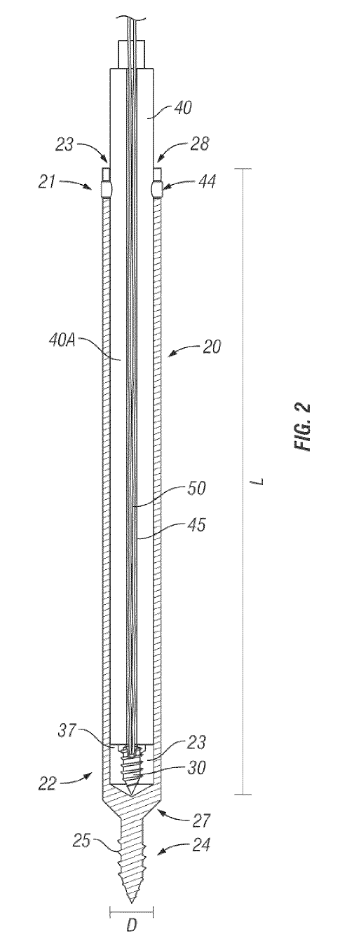

As shown in FIGS. 1 and 2, removable tap sleeve (20) comprises a longitudinally-extending blind bore (23), with opening at proximal end (21) and extending toward the tap sleeve distal end (22), a shoulder (27), and a tap (24). Blind bore (23) bears a diameter and a length sufficient to removably accommodate a shaft (40), bearing at shaft first end (40A) suture anchor (30) or any other suitable device designed to attach to a suture, and at least one length of suture (50) extending from the anchor (30) to the handle (70). Tap sleeve proximal end (21) further comprises notches (28) keyed to engage with complementarily keyed posts (44) of shaft (40) as shaft first end (40A), with or without anchor (30) and at least one suture (50), is reversibly inserted into blind bore (23). Tap sleeve distal end (22) comprises shoulder (27) and cylindrical tap (24), the most distal portion of which tapers to a point. Tap (24) comprises threads (25) and cutting flute (26). The threads (25) of tap (24) are selected to be compatible with the threads (36) of the anchor (30), and the bone hole produced by tap (24) is designed to be suitable for installation of anchor (30). The tap sleeve (20) can be made of materials including, but not limited to, stainless steel, titanium, and titanium alloy. By inserting the shaft first end (40A), with or without anchor (30) and at least one suture (50), into the blind bore (23) of the tap sleeve (20) so that the posts (44) of the shaft (40) interlock with the notches (28) of the tap sleeve (20), rotational force may be imparted manually via the handle (70) to the tap (24) when the shaft second end (40B) is attached to anti-rotation sleeve element (77) and installed in the handle (70) central void (86).

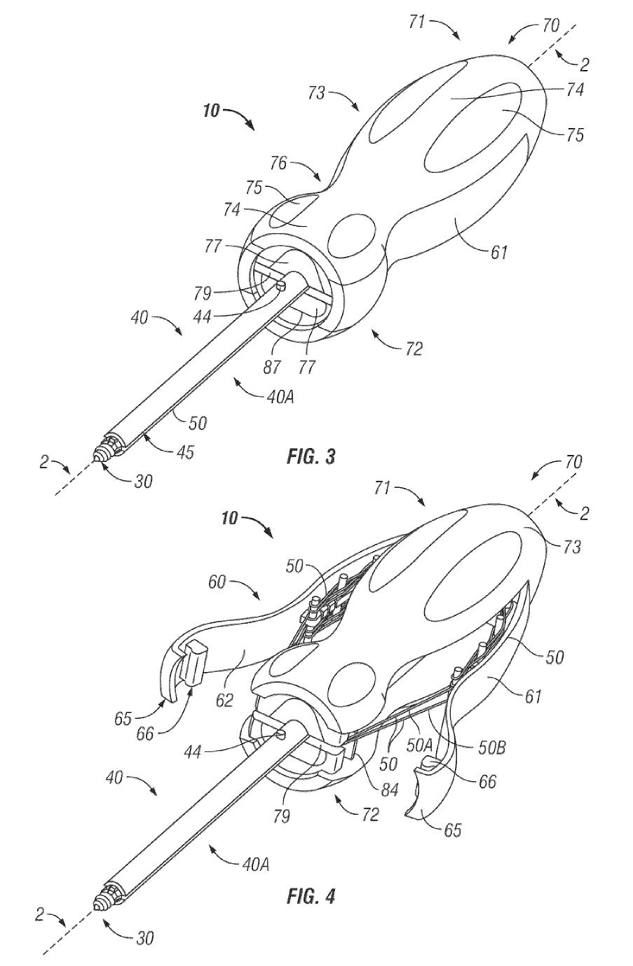

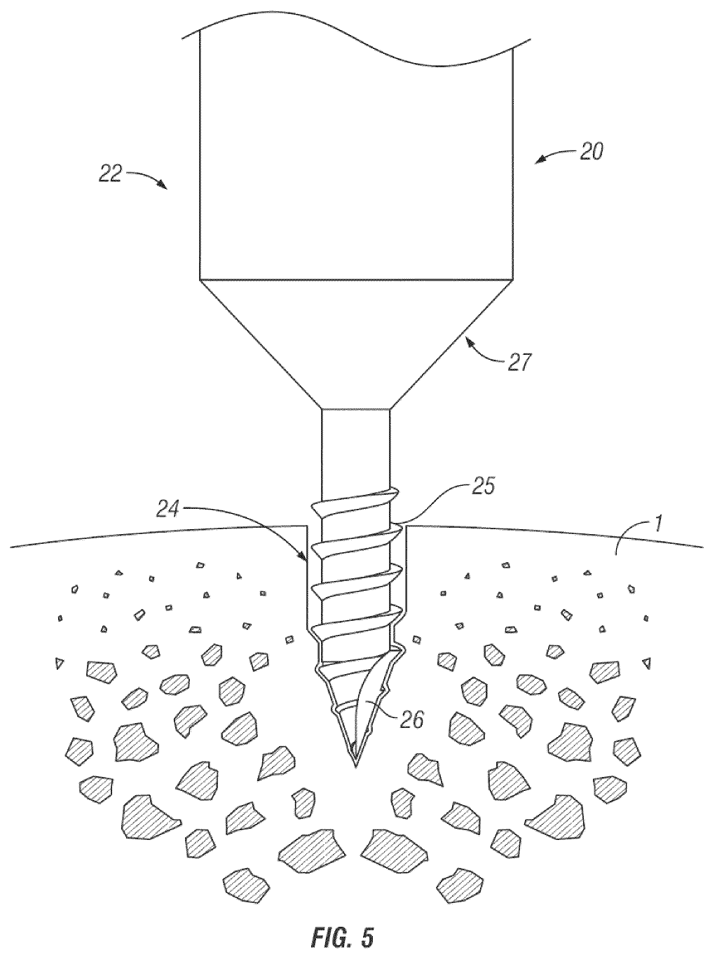

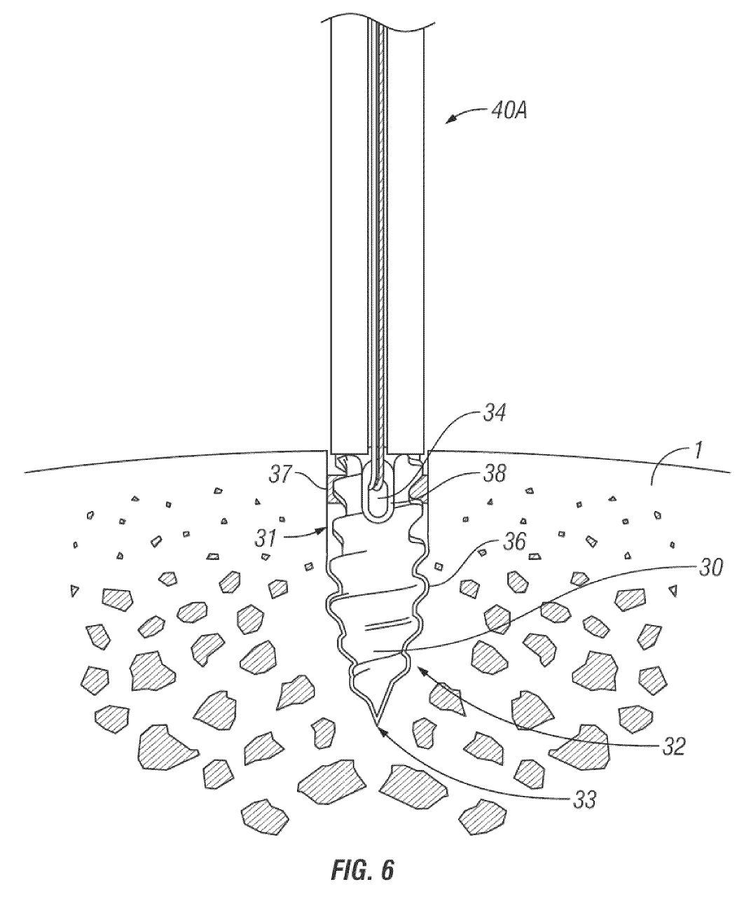

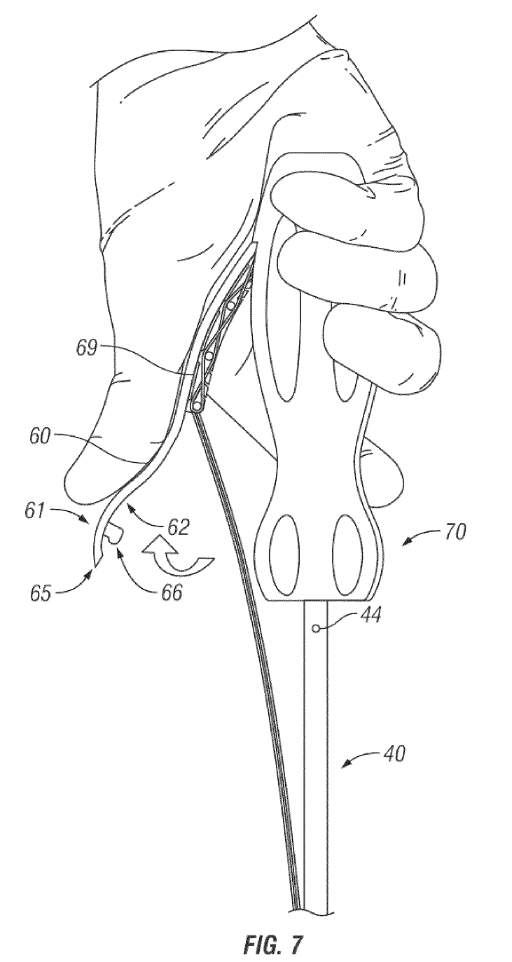

As shown in FIGS. 1, 3, and 4, handle (70) of instrument (10) comprises an outer surface (73) provided with a grip formed of a waist (76) disposed between proximal (71) and distal (72) ends, and alternating raised edges (74) and depressions (75) bisected by waist (76). In the preferred embodiment, outer surface (73) is provided with a grip formed of four alternating raised edges (74) and depressions (75), bisected by waist (76). In this configuration, a user holding the handle can grasp the instrument (10) firmly and—as described more fully below—may easily produce a threaded hole in a bone (1), as shown in FIG. 5, and insert a suture anchor (30), as shown in FIG. 6.

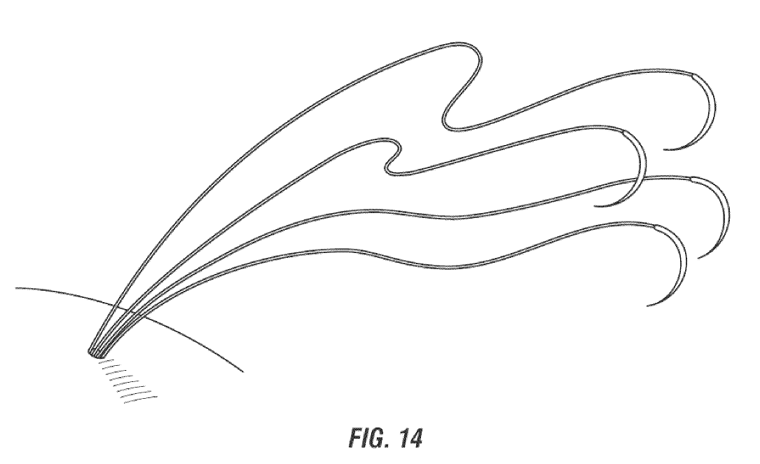

As shown in FIG. 4, handle (70) is provided with at least one cavity (81) for housing suture (50), or suture (50) attached to at least one needle (53). Optionally, a plurality of sutures (50A, 50B) with or without needles (53) may be housed within the at least one cavity (81). In the preferred embodiment, the handle (70) is provided with two cavities (81) disposed approximately opposite one another (as shown in FIG. 4). In this configuration, a first suture end of a first suture (50A) is housed in one of the two cavities (81), and the second suture end of said first suture (50A) is housed in the opposing cavity (81). Also in this configuration, and simultaneously with first suture (50A), a first suture end of a second suture (50B) may be housed in one of the two cavities (81) and the second suture end of said second suture (50B) may be housed in the opposing cavity (81). Further in this configuration, and at the user's option, either suture end, both ends, or neither end of either first or second sutures (50A, 50B) may bear a needle (53) (see, e.g., FIG. 14). In an alternative embodiment, and as will be recognized by those of ordinary skill in the relevant art, the handle (70) may be provided with a single cavity (81), in which event the first suture ends and the second suture ends of each suture (50A, 50B) would each be housed in the same cavity (81) along with any needles (53) attached to said ends.

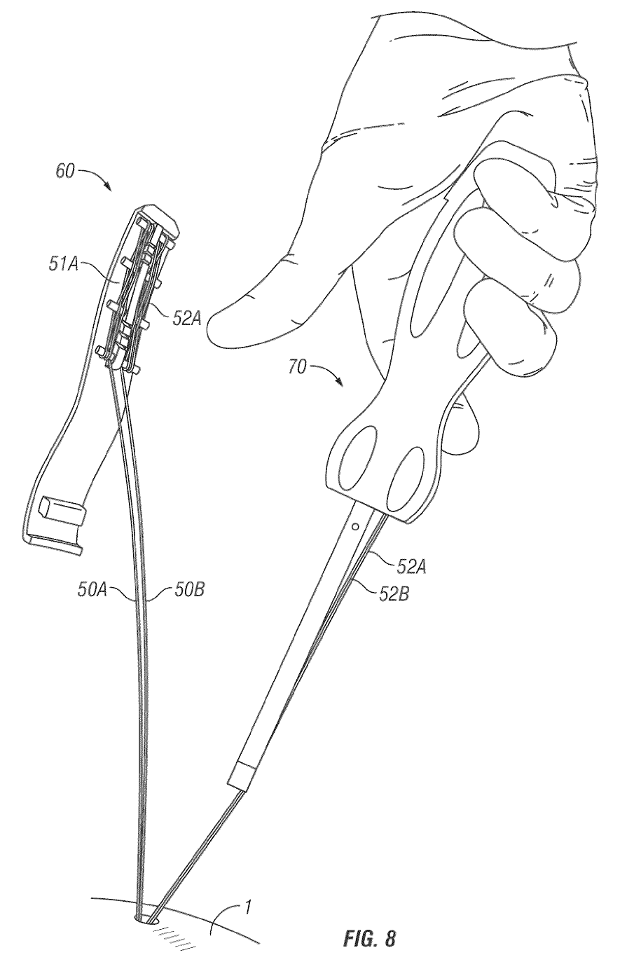

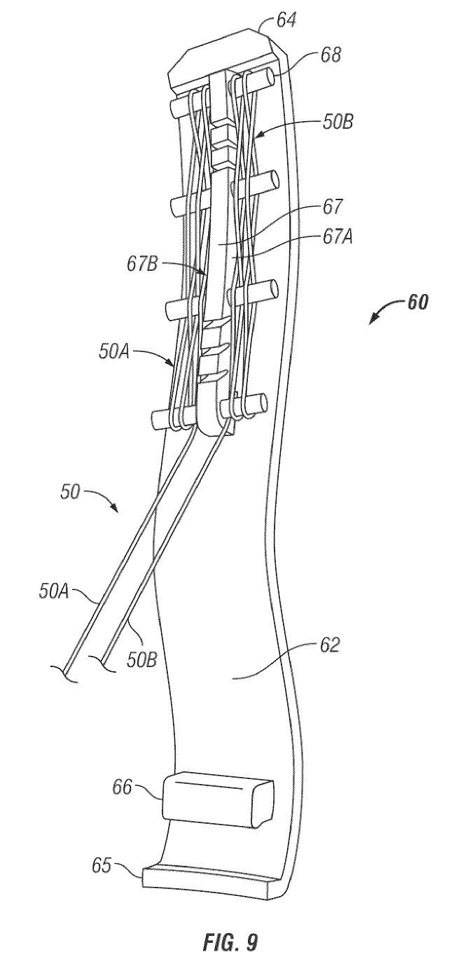

Also as shown in FIGS. 4, 7, and 8, each cavity (81) is accessed via a pivotable, removable door (60). Each door has an outer surface (61), the outer surface (61) lying substantially flush with the outer surface (73) of handle (70) when the door (60) is closed. Each door also has an inner surface (62), shown more clearly in FIG. 5, which (when the door is closed) forms a boundary of a cavity (81) in the handle (70). Door (60) further comprises tongue (64), lip (65), and latch (66). Lip (66) is coextensive with the most distal end of door (60), projects substantially radially toward the longitudinal axis (2) and the shaft (40), and provides convenient means for a user of the instrument (10) to manipulate the door (60). Latch (66) lies proximal to lip (65), on the door inner surface (62), and is configured to operate in concert with strike (84) contained within the cavity (81) of handle (80). Upon closing door (60), latch (66) engages strike (84) to securely but releasably hold door (60) in a closed configuration. Tongue (64) is coextensive with the most proximal end of door (60), and is guided into groove (83) contained within the cavity (81) and configured to receive the tongue (64) by shelf (82).

FIGS. 7-9 depict the inner surface (62) of door (60), which further comprises longitudinally projecting rib (67), extending substantially along longitudinal axis (2). The rib (67) supports a plurality of niches (69; see, e.g., FIG. 7) and cleats (68). Each cleat (68) lies across and substantially perpendicular to the rib (67), and elevated from the door inner surface (62). The cleats (68) are shown in FIG. 9 as elongated cylinders projecting substantially perpendicular to rib (67), but those of ordinary skill in the art will recognize that other geometries are possible. Each niche (69) pierces the rib (67) between the door inner surface (62) and the cleats (68), thus forming a passage through the rib (67), whereby each niche (69) may provide convenient means for receiving, organizing, and storing needles (53). In the preferred embodiment, the inner surface (62) of each pivotable, removable door (60) is provided with one rib (67), the rib (67) supporting four niches (69) and four cleats (68). In this configuration, a first suture end of a first suture (50A) is threaded about at least one cleat (68), and a first suture end of a second suture (50B) is threaded about at least one cleat (68). Preferably, the first and second sutures (50A, 50B) are each threaded onto opposite sides of the rib (67), but may or may not be threaded about the same cleat (68). More preferably, a first suture end of a first suture (50A) is threaded about all cleats (68) in a “figure eight” pattern, on one side of the rib (67). Then, optionally, a first suture end of a second suture (50B) is threaded about all cleats (68) in a “figure eight” pattern, on the opposite side of the rib (67). As would be appreciated by a person having ordinary skill in the art, the number of niches (69) and cleats (68), as well as the size and orientation thereof, is susceptible of variation in order to accommodate various suture thicknesses and needle sizes or geometries. As shown in FIG. 9, for example, the rib (67) may optionally have a plurality notches cut into it and extending toward the door inner surface (62). Preferably, these notches are omitted.

The handle, handle doors, and all other components of the handle (for example, but not limited to, anti-rotation sleeve element (77), cleats (68), etc.) may be made of materials including, but not limited to, acrylonitrile butadiene styrene (“ABS”), polyphenylsulphone (“PPSF” or Radel®), or Ultem®.

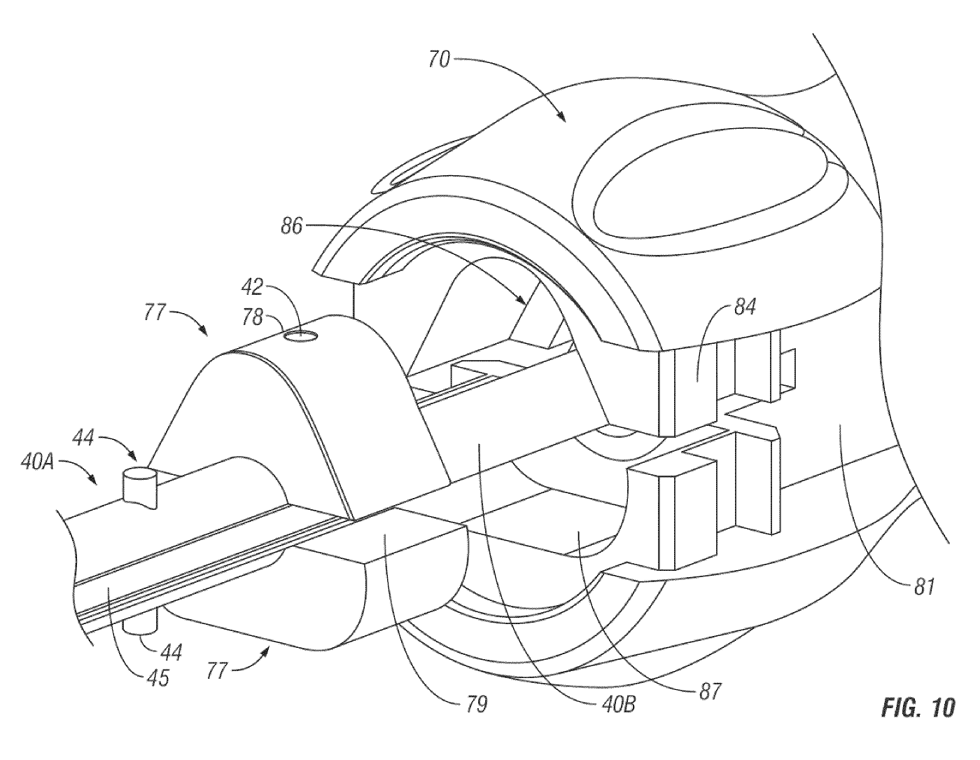

The handle (70) is adapted for releasable engagement with both the first end (40A) and the second end (40B), as desired, of shaft (40), as shown in FIG. 10. The handle (70) includes a central void (86) extending axially therethrough to accommodate a portion of the shaft (40). Handle (70) is preferably adapted to receivingly engage with an anti-rotation sleeve element (77), which is insertable into an opening (87) in the handle and which is adapted for engagement with the shaft (40). In the illustrated embodiment, the anti-rotation sleeve element (77) has a triangular cross-section, and fits within a corresponding triangular bore (87) in the handle (70). This non-circular design facilitates the secure engagement of the shaft (40) in the handle (70) and permits the handle (70) and shaft (40) to be rotated together in both the clockwise and counter-clockwise directions without slipping relative to one another. The anti-rotation sleeve element (77) has a through bore (78) adapted to receive a pin (42) that penetrates the shaft (40) and so secures the sleeve element (77) to the shaft (40). The sleeve element (77) also has two suture slots (79), each substantially parallel to furrows (45) of shaft first end (40A), whereby suture (50) extending from a suture anchor (30) may communicate with the handle cavities (81) (see FIGS. 7-9) and structures therein.

The second end (40B) of the shaft (40) fits within the central void (86) of the handle (70), as shown in FIG. 10. The shaft second end (40B) can be inserted into the handle by pushing it axially into the central void (86) until the triangular sleeve element (77) engages with the correspondingly-shaped triangular bore (87) in the handle. Similarly, the shaft (40) can be easily removed from the handle (70) by pulling it axially from the handle until the sleeve element (77) releases from the handle. The first end (40A) of the shaft (40) also fits within the central void (86) of the handle (70), and can also be inserted into the handle by pushing it axially into the central void (86) until the triangular sleeve element (77) engages with the correspondingly-shaped triangular bore (87) in the handle. This arrangement permits the shaft first end (40A) to have a functionality and the shaft second end (40B) to have a functionality which may be the same as or different from the functionality of the shaft first end (40A).

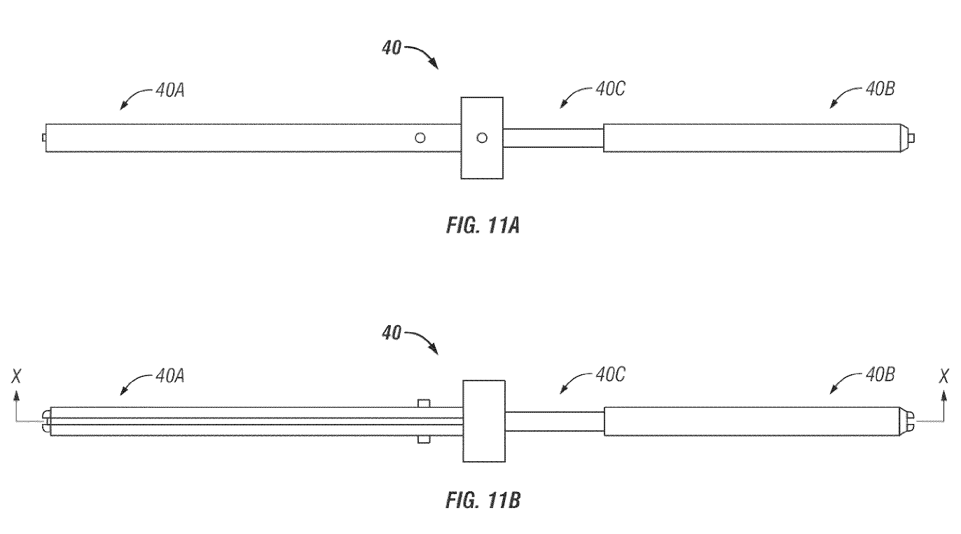

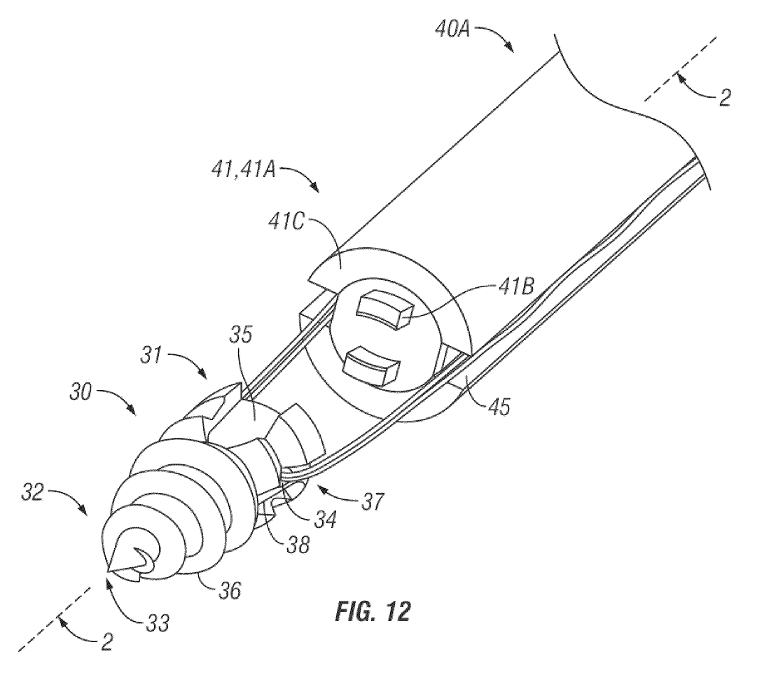

Instrument (10) also comprises shaft (40), illustrated in greater detail in FIGS. 11A and 11B, with a shaft second end (40B) normally disposed within a central void (86) of handle (70), a shaft first end (40A), and a shaft middle (40C) therebetween. As shown in FIGS. 11A, 11B, and 12, shaft first end (40A) comprises installation prongs (41B) at first end distal tip (41A), said prongs (41B) keyed to engage with complementarily keyed channels (35) in drive head (37) of suture anchor (30). As shown, shaft first end (40A) bears two installation prongs (41B), opposed to one another across the shaft diameter and projecting coaxially with the longitudinal axis (2). Installation prongs (41B) are keyed to the channels (35) of anchor (30), and insertion of prongs (41B) into the channels (35) by mating the anchor drive head (37) to the shaft first end distal tip (41A) enables a user to rotate the anchor (30) and thus turn the anchor (30) into or out of an installation site in a bone (1) (see, e.g., FIG. 6). Installation prongs (41B) are shaped and separated from one another so that the drive head (37) of anchor (30) may be supported by and engaged with those prongs (41B), the prongs (41B) fitting within and engaging the channels (35) of anchor (30) to enable the anchor to be rotated and thus turned into or out of an installation site in a bone. In a preferred embodiment, each prong (41B) includes a chamfer of about 25 to about 35 degrees. The chamfers preferably extend in opposite directions to facilitate entry of the anchor (30) into the bone.

As shown in FIGS. 11A and 11B, shaft second end (40B) comprises removal prongs (49B) at second end distal tip (49A), said prongs (49B) keyed to releasably engage with complementarily keyed channels (35) in drive head (37) of a suture anchor (30). The second end distal tip (49A) is designed to facilitate removal or loosening of the suture anchor (30) from its installation site in a bone, should such removal or loosening be desired. Shoulder (49C) of shaft second end (40B) lies distal (i.e., lies toward shaft first end (40A)) to the removal prongs (41B). The most proximal portion of the shoulder (49C) establishes the diameter of the flat face of second end distal tip (49A), while the most distal portion of the shoulder (49C) corresponds to the diameter of the shaft second end (40B). The flat face of the second end distal tip is substantially perpendicular to the longitudinal axis (2). In a preferred embodiment, the second end distal tip (49A) of the shaft second end (40B) includes a pair of removal prongs (49B), which extend into and engage with the channels (35) of the drive head (37) of suture anchor (30). Counterclockwise rotation of the shaft second end (40B) in the drive head (37) of a suture anchor (30) which has been installed into a bone operates to disengage the anchor (30) from the bone and permits it to be loosened or removed. Because the shaft second end (40B) bears a relatively gradual taper from shaft diameter to second end distal tip (49A) diameter, via shoulder (49C), removal prongs (49B) may reach deeper into a hole within a bone than if said shoulder (49C) were absent. Thus, shaft second end (40B) may engage more securely with the channels (35) of a suture anchor (30) to be removed. The more gradual taper of the shaft second end (40B) also helps to prevent unwanted contact between the shaft second end (40B) and the bone hole, which may inadvertently enlarge or otherwise damage the hole as an anchor (30) is removed.

In a preferred embodiment, both the shaft first end (40A) and the shaft second end (40B) are adapted to fit within the central void (86) of the handle (70), as selected by the user. With this design, the shaft first end (40A) can include installation prongs (41B) and the shaft second end (40B) can include removal prongs (49B) opposite the installation prongs (41B). As desired by the user, either end (40A, 40B) of the shaft (40) can be engaged securely with the handle (70) via anti-rotation sleeve element (77) and triangular bore (87).

As shown in FIGS. 2, 3, 4, 6, 10, and 12, the shaft first end (40A) further comprises a pair of furrows (45) extending longitudinally along either side of the shaft (40), from the first end distal tip (41A) and into each handle cavity (81). The furrows are disposed to align with the recesses (38) of the anchor (30) and provide sufficient width and depth to provide a safe avenue for suture (50) threaded through the anchor eyelet (34) to pass along the shaft (40) and into the handle cavity (81) without becoming abraded by (for example) the inner wall of the tap sleeve bore (23).

The shaft (40) may be made of materials including, but not limited to, stainless steel, titanium, and titanium alloy.

As seen in FIGS. 1-4, 11A, and 11B, the instrument (10) further includes cylindrical posts (44) for releasable engagement with tap sleeve (20) via notches (28). The posts (44) are located proximal to the shaft first end (40A) and distal to the shaft second end (40B), and project radially from the longitudinal axis (2) for a distance sufficient to securely but releasably engage tap sleeve notches (28) without interfering unduly with normal operation of the device (10) for its intended purposes. In a preferred embodiment, notches (28) of tap sleeve (20) each have a length coaxial to the instrument longitudinal axis (2) and a width substantially perpendicular to the longitudinal axis (2). In this preferred embodiment, the cylindrical posts (44) each substantially define a cylinder—a surface formed by the points at a fixed distance from an imaginary straight line that projects perpendicular to the longitudinal axis (2), the surface being capped by a plane parallel to the longitudinal axis (2). The length of the straight line between the point at which it would meet the shaft exterior surface and the point at which the cylinder is capped by the plane parallel to the longitudinal axis defines the height of each post (44). Each cylindrical post (44) has a height and a diameter sufficient and appropriate to securely but releasably engage tap sleeve notches (28), and preferably a height sufficient for the post ends to sit substantially flush with the tap sleeve (20), when installed. In practice, a hole may be drilled through shaft (40) and perpendicular to longitudinal axis (2) to accommodate the installation of a single cylindrical post (44), said post (44) having a length sufficient to project through the shaft (40) radially from the longitudinal axis (2) and securely but releasably engage tap sleeve notches (28).

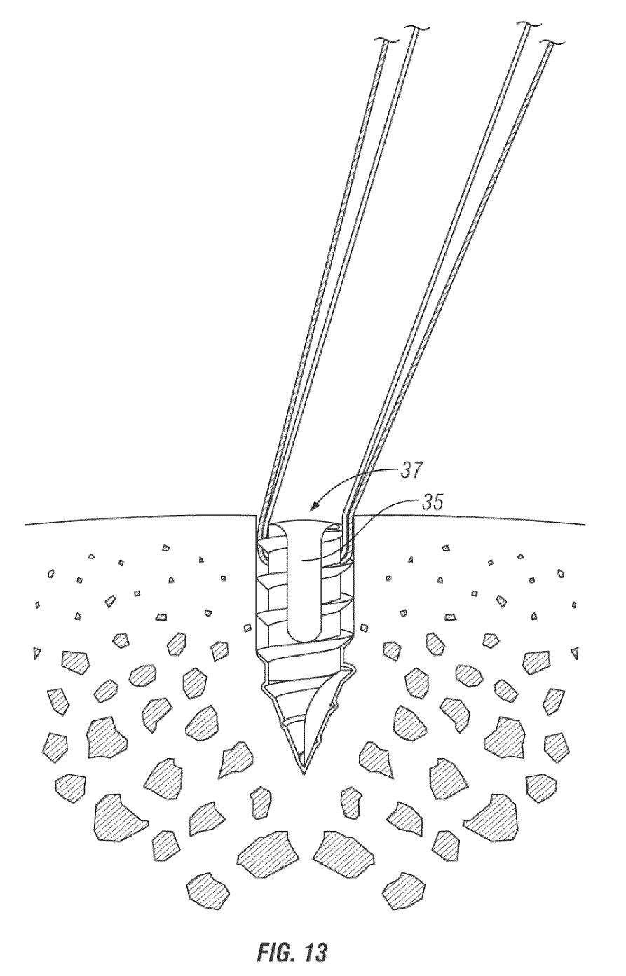

As shown in FIGS. 2-4, and 12, instrument (10) further comprises suture anchor (30). The suture anchor (30) is of a type known in the art which extends coaxially with longitudinal axis (2), between an anchor tip (33) at one end and a drive head (37) at an opposite end. The suture anchor (30) is threaded along at least a portion of its length or otherwise adapted for secure engagement in bone, and includes an eyelet (34) in the drive head (37). The eyelet (34) is sufficiently large and suitably finished to accommodate one or more lengths of a suture (50) or sections of soft tissue. In the illustrated embodiment, the suture anchor (30) is threaded over the entire length of the outside surface of the anchor from the anchor tip (33) to the drive head (37), as shown in FIGS. 2-4, and 12. The eyelet (34) of the illustrated suture anchor is the so-called “inverted” type which extends transversely through the body of the suture anchor near the drive head (37) of the anchor (30). As shown in FIGS. 12 and 13, the anchor (30) includes a pair of channels (35), with the center of each channel approximately 90 degrees from the center of each eyelet (34). Each channel (35) lies in a direction parallel to and coaxial with the longitudinal axis (2). The channels (35) permit a two-pronged shaft first end (40A) or two-pronged shaft second end (40B) to engage with the drive head (37) of the anchor, as detailed more fully above. The anchor (30) also includes a pair of recesses (38) extending on either side of the eyelet (34) in a direction parallel to the longitudinal axis (2) (see FIGS. 6 and 12). The recesses (38) permit the sutures (50) to pass upward, within the recesses (38), into the shaft furrows (45), and also reduce the likelihood of the suture (50) being sheared by the anchor threads (36) as the anchor (30) is driven into a bone.

The suture anchor (30) may be made of materials including, but not limited to, titanium alloy (e.g., Ti6Al4V), and plastics (e.g., PLLA, PLDLA, PLA/PGA, PEEK, and PLLA/TCP blends). As used herein, “PLLA” is poly-L-lactide, “PLDLA” is poly-L-co-D-L-lactide, “PLA” is polylactide, “PGA” is polyglycolic acid, “PEEK” is polyetheretherketone, and “TCP” is tricalcium phosphate.

FIGS. 6, 12, and 13 are meant only to be representative of the applicability of the present invention and are by no means complete with regard to the anchor type, the engagement geometry, or the number of suture passages through an anchor. For example, those of ordinary skill in the art will appreciate that the engagement geometry between distal shaft and drive head as shown in FIGS. 6, 11A, 11B, 12, and 13 may be adapted to be used with virtually any anchor type, including but not limited to those shown in: U.S. Pat. Nos. 4,537,185; 5,571,139; 6,511,499; 6,648,892; 7,163,540; 7,322,978; and WO 2008/054814, herein incorporated by reference in their entireties. Furthermore, engagement between a shaft and an anchor may be between an external cross-section (i.e., male) of the shaft and an internal cross-section (i.e., female) of a drive head of an anchor. Those of ordinary skill in the art will also recognize that the engagement geometry between posts (44) and notches (28) may be adapted to permit different configurations (e.g., a post with square cross-section, or modifying notch (28) to provide an “L” shape to further secure tap sleeve (20) to shaft (40)).

The shaft first end (40A) and the shaft second end (40B), as well as the suture anchor, suture, and needles, are all provided in a variety of sizes to accommodate the various sizes required for various surgical procedures. The at least one suture (50) can be made of materials including, but not limited to, polyethylene, ultra high molecular weight polyethylene (“UHMWPE,” also known as “high-modulus polyethylene,” “high performance polyethylene,” and “ultra high molecular weight polyethylene”) and woven UHMWPE.

A suture anchor can be conveniently installed into a bone and threaded with a suture or a section of tendon or other soft tissue after installation according to the following method. First, a suture anchor of the appropriate size is selected. A corresponding shaft bearing an appropriate shaft first end (40A) and an appropriate shaft second end (40B) are then selected. An anti-rotation sleeve element is disposed around the shaft and secured with a pin. A handle which is suitable for engaging the shaft first end (40A) and the shaft second end (40B) is then selected. The handle is assembled to the driver shaft by sliding the handle over the shaft and aligning the anti-rotation sleeve on the shaft with the corresponding opening in the handle, with the shaft first end (40A) of the shaft (40) exposed and the shaft second end (40B), with removal prongs (49B), if provided, disposed within the interior of the handle.

The suture anchor (30) may then be loaded onto the shaft (40) of the instrument (10) as follows. The free ends of at least one working or repair suture (50A) is threaded through the eyelet (34) disposed at the drive head (37) of suture anchor proximal end (31). Preferably, the free ends of two separate working or repair sutures (50A, 50B) are threaded through the eyelet (34), and the remainder of this description will describe the use of two sutures. The channels (35) of the anchor (30) are then aligned with the installation prongs (41B) and the recesses (38) are aligned with the shaft furrows (45). The first free suture ends are then guided along a shaft furrow (45) and through a suture slot (79) of sleeve element (77), toward a cavity (81) of the handle (70). The second free suture ends are guided along the opposite shaft furrow (45) and through the opposite suture slot (79) of sleeve element (77), toward the opposite cavity (81) of the handle (70). The first free suture end of a first suture (50A) is threaded about all cleats (68) in a “figure eight” pattern, on one side of the rib (67) of one door (60), and the first free suture end of the second suture (50B) is threaded about all cleats (68) in a “figure eight” pattern, on the opposite side of the rib (67) of the same door (60) used for the first free end of the first suture (50A). Similarly, the second free suture end of the first suture (50A) is threaded about all cleats (68) in a “figure eight” pattern, on one side of the rib (67) of the opposite door (60), and the second free suture end of the second suture (50B) is threaded about all cleats (68) in a “figure eight” pattern, on the opposite side of the same rib (67), of the same door (60) used for the second free suture end of the first suture (50A). If the sutures (50A, 50B) comprise needles (53), they may be mounted within the appropriate niches (69). Then, upon inserting the tongue (64) of a door (60) into an appropriate groove (83) of a handle cavity (81), each door may be pivoted about its tongue (64) to bring the corresponding latch (66) against the corresponding strike (84) and thereby fasten each door closed so that it is substantially flush with the handle (70). Tension on the sutures (50A, 50B) is maintained via a silicone sleeve (not shown) positioned inside of the handle (70), behind the sleeve element (77), and is concentric about and attached to the shaft (40). The sutures (50A, 50B) proceed from the anchor (30), up the furrows (45) in the shaft (40), through the suture slots (79) of the sleeve element (77), and under the silicone sleeve (not shown). Sutures passed between the shaft (40) and the concentric silicone sleeve (not shown) then enter the handle cavity (81) and are attached to the doors (60) via cleats (68). Finally, the shaft (40) bearing anchor (30) secured to it via suture (50A, 50B) is removably inserted into the blind bore (23) of tap sleeve (20) so that the shaft posts (40) interlock with the tap sleeve notches (28).

In this configuration, the instrument may be sterilized via suitable techniques known in the art (for example, without limitation, via autoclaving, gamma irradiation, and ethylene oxide).

In use, the distal tip of the tap (24) may be placed against the bone into which it is to be installed (e.g., it may be placed substantially perpendicular to said bone). The instrument (10) is rotated clockwise via the handle, with application of constant axial pressure. Care should be taken to keep the tap and tap sleeve perpendicular to the bone to prevent inadvertent dissociation of the tap from the bone. The tap sleeve is rotated in a clockwise direction until an appropriate depth is reached, after which the tap sleeve is rotated in a counterclockwise direction until it disengages from the bone. The tap sleeve is then removed from the instrument, revealing the anchor attached to the shaft.

The distal end (32) or tip of the anchor (30) is then placed substantially in alignment with the bone hole formed by the tap. The shaft (40) is rotated clockwise by the handle (70) with application of constant axial pressure. Care should be taken to keep the anchor perpendicular to the bone to prevent premature dissociation of the anchor from the shaft first end (40A). The shaft is rotated in a clockwise direction until the anchor disengages from the shaft first end (40A) and is fully installed in the bone. After disengagement, the handle doors may be opened and removed from the handle, thereby freeing the handle and attached shaft to be removed from the surgical site. The sutures, threaded through the suture anchor and having their first and second ends retained and organized by the cleats on the inner surface of each door, remain. Once disengaged from the cleats, the sutures are free to be employed at the user's discretion (FIG. 14).

Should the user determine that the anchor must be loosened, disengaged, or even fully removed from its location in the bone, the shaft second end (40B) may be removed from the handle (70) and the shaft first end (40A) inserted into said handle. Then, using the removal prongs (49B) of the shaft second end (40B) and taking advantage of the deeper reach afforded the removal prongs (49B) by the shoulder (49C), the removal prongs (49B) are aligned with and inserted into the channels (35) of the anchor (30). Then, by counterclockwise rotation of the shaft (40) with application of constant axial pressure, the anchor may be reversed, thereby loosening, disengaging, or even fully removing the anchor from the bone hole.

TABLE 1 is provided below for the reader's convenience, listing the enumerated elements described above:

TABLE 1

| No. | Description |
|---|---|
| 1 | Bone |
| 2 | Longitudinal axis |
| 10 | Instrument |
| 20 | Tap sleeve |
| 21 | Tap sleeve proximal end |
| 22 | Tap sleeve distal end |
| 23 | Blind bore |
| 24 | Tap |
| 25 | Tap threads |
| 26 | Cutting flute |
| 27 | Shoulder |
| 28 | Notch |
| 30 | Anchor |
| 31 | Anchor proximal end |
| 32 | Anchor distal end |
| 33 | Anchor tip |
| 34 | Eyelet |
| 35 | Channel |
| 36 | Anchor threads |
| 37 | Drive head |
| 40 | Shaft |
| 40A | Shaft first end |
| 40B | Shaft second end |
| 40C | Shaft middle |
| 41A | First end distal tip |
| 41B | Installation prong |
| 42 | Pin |
| 44 | Post |
| 45 | Furrow |
| 49A | Second end distal tip |
| 49B | Removal prongs |
| 49C | Second end shoulder |
| 50 | Suture |
| 50A | First suture |
| 50B | Second suture |
| 53 | Needle |
| 60 | Door |
| 61 | Door outer surface |
| 62 | Door inner surface |
| 64 | Tongue |
| 65 | Lip |
| 66 | Latch |
| 67 | Rib |
| 68 | Cleat |
| 69 | Niche |
| 70 | Handle |
| 71 | Handle proximal end |
| 72 | Handle distal end |
| 73 | Handle outer surface |
| 74 | Raised edge |
| 75 | Depression |
| 76 | Waist |
| 77 | Sleeve element |
| 78 | Sleeve element bore |
| 79 | Suture slot |
| 80 | Inner surface |
| 81 | Cavity |
| 82 | Shelf |
| 83 | Groove |
| 84 | Strike |
| 86 | Central void |
| 87 | Triangular bore |

All references cited in this specification are herein incorporated by reference as though each reference was specifically and individually indicated to be incorporated by reference. The citation of any reference is for its disclosure prior to the filing date and should not be construed as an admission that the present invention is not entitled to antedate such reference by virtue of prior invention.

It will be understood that each of the elements described above, or two or more together may also find a useful application in other types of methods differing from the type described above. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only; the scope of the present invention is to be limited only by the following claims.

The invention claimed is:

1. An apparatus for inserting surgical implants, comprising:
a) a handle;
b) a shaft extending along a longitudinal axis between first and second ends, wherein the shaft second end engages the handle;
c) an anchor extending along a longitudinal axis between proximal and distal ends, wherein the anchor proximal end engages the shaft first end; and
d) a tap sleeve extending along a longitudinal axis between proximal and distal ends, the tap sleeve having a blind bore with an opening at said tap sleeve proximal end and extending longitudinally toward said tap sleeve distal end, and the tap sleeve distal end comprising a tap;
wherein the tap sleeve removably accommodates said anchor and at least a portion of the shaft first end via the tap sleeve blind bore.

2. The apparatus of claim 1, wherein the shaft further comprises at least one post.

3. The apparatus of claim 2, wherein said at least one post is substantially cylindrical.

4. The apparatus of claim 2, wherein the tap sleeve further comprises at least one notch, wherein said at least one notch is keyed to engage said at least one post.

5. The apparatus of claim 4, wherein said shaft comprises first and second posts and said tap sleeve comprises first and second notches, and wherein said first and second notches are keyed to engage said first and second posts.

6. The apparatus of claim 1, further comprising at least one suture having first and second ends, wherein:
a) said anchor further comprises threads, an eyelet, and at least one channel;
b) said shaft first end comprises at least one installation prong;
c) said at least one suture passes through said eyelet; and
d) said at least one channel and at least one installation prong are keyed to engage one another.

7. The apparatus of claim 6, wherein said handle comprises at least one cavity and at least one door, the at least one door comprising attachment means for said at least one suture.

8. The apparatus of claim 7, wherein the attachment means for said at least one suture comprise a plurality of cleats arranged to engage said at least one suture.

9. The apparatus of claim 8, wherein said plurality of cleats are located on the door inner surface.

10. The apparatus of claim 9, wherein said at least one door is releasably engaged with said handle.

11. The apparatus of claim 10, wherein:
a) the handle comprises first and second cavities and first and second doors; and
b) the first suture end is engaged with the plurality of cleats of the first door, and the second suture end is engaged with the plurality of cleats of the second door.

12. The apparatus of claim 11, wherein:
a) the first suture end further comprises a first needle attached thereto, and the second suture end further comprises a second needle attached thereto; and
b) the first and second needles are located in the first and second cavities, respectively.

13. The apparatus of claim 12, wherein:
a) said shaft further comprises at least one furrow extending substantially from said shaft first end and toward shaft second end, whereby said at least one suture is directed toward the handle; and
b) said handle further comprises at least one suture slot, whereby said at least one suture communicates with said at least one cavity.

14. The apparatus of claim 13, wherein:
a) said shaft comprises first and second furrows extending substantially from said shaft first end and toward shaft second end, whereby the first and second suture ends are directed along and within said first and second furrows, respectively, toward the handle; and
b) said handle comprises first and second suture slots, whereby the first suture end communicates with the first cavity and the second suture end communicates with the second cavity.

15. The apparatus of claim 14, wherein:
a) said shaft second end is removably engaged with said handle;
b) said shaft first end may removably engage with said handle;
c) said shaft second end further comprises at least one removal prong keyed to engage said at least one channel.

16. An apparatus for inserting surgical implants, comprising:
a) a handle, comprising first and second cavities and first and second doors releasably engaged with the handle, each door comprising a plurality of cleats on the door inner surface, said handle further comprising first and second suture slots;
b) a shaft extending along a longitudinal axis between first and second ends, wherein the shaft second end removably engages the handle and wherein the shaft first end may removably engage with the handle, wherein the shaft comprises first and second posts extending substantially perpendicular to the shaft longitudinal axis, wherein the shaft first end comprises first and second installation prongs, the shaft second end comprises first and second removal prongs, the shaft further comprises first and second furrows extending substantially from said shaft first end and toward shaft second end;
c) an anchor extending along a longitudinal axis between proximal and distal ends, comprising threads, an eyelet, and first and second channels, wherein the anchor proximal end engages the shaft first end, and the first and second channels and first and second installation prong are keyed to engage one another;
d) at least one suture having first and second ends, wherein the at least one suture passes through the eyelet, the first suture end further comprises a first needle and is engaged with the plurality of cleats of the first door, the second suture end further comprises a second needle and is engaged with the plurality of cleats of the second door, whereby the first and second needles are located in the first and second cavities, respectively; and
e) a tap sleeve extending along a longitudinal axis between proximal and distal ends, the tap sleeve having a blind bore with an opening at said tap sleeve proximal end and extending longitudinally toward said tap sleeve distal end, the tap sleeve distal end comprising a tap, the tap sleeve proximal end comprising first and second notches keyed to engage first and second posts;
whereby the first and second suture ends are directed along and within said first and second furrows, respectively, toward the handle, whereby the first suture end communicates with the first cavity and the second suture end communicates with the second cavity, wherein the tap sleeve removably accommodates said anchor, said at least one suture, and at least a portion of the shaft first end via the tap sleeve blind bore.

17. A method for attaching soft tissue to bone, comprising the steps of:
a) providing an apparatus comprising:
i) a handle, comprising first and second cavities and first and second doors releasably engaged with the handle, each door comprising a plurality of cleats on the door inner surface, said handle further comprising first and second suture slots;
ii) a shaft extending along a longitudinal axis between first and second ends, the shaft second end removably engaged with the handle;
iii) an anchor extending along a longitudinal axis between proximal and distal ends and comprising an eyelet, wherein the anchor proximal end engages the shaft first end;
iv) at least one suture having first and second ends, the suture being threaded through the eyelet, the first suture end comprising a first needle and the second suture end comprising a second needle, wherein the first suture end is engaged with the plurality of cleats of the first door and the second suture end is engaged with the plurality of cleats of the second door; and
v) a tap sleeve extending along a longitudinal axis between proximal and distal ends, wherein the distal end comprises a tap, the tap sleeve further comprising a blind bore extending along the longitudinal axis, the tap sleeve removably attached to the shaft first end and accommodating the anchor, at least a portion of the at least one suture, and at least a portion of the shaft first end;
b) forming a threaded hole in a bony structure with the tap of the tap sleeve removably attached to the shaft first end;
c) removing the tap sleeve from the shaft first end;
d) inserting the anchor into the threaded hole; and
e) attaching soft tissue to said suture.
